(19) United States
(12) Patent Application Publication
(10) Pub. No.: US 2010/0183722 A1
(43) Pub. Date: Jul. 22, 2010

(54) MODULATION OF PROTEASES. PARTICULARLY IN THE TREATMENT OF CHRONIC ULCEROUS SKIN LESIONS

(76) Inventor: Hugh Semple Munro, Chipping Campden (GB)

Correspondence Address: FISH & RICHARDSON P.C., PO BOX 1022, MINNEAPOLIS, MN, 55440-1022, US

(21) Appl. No.: 12/523,717

(22) PCT Filed: Jan. 17, 2008
(86) PCT No.: PCT/GB2008/000147

§ 371 Date: Mar. 26, 2010

(30) Foreign Application Priority Data
Jan. 17, 2007 (GB) 0700908.7

(51) Int. Cl. A61K 9/00; A61P 17/02; A61K 31/74

(52) U.S. Cl. 424/484; 424/78.06

(57) ABSTRACT

The invention provides a method of modulating proteases, particularly when treating a wound (for example, a chronic ulcerous skin lesion) in a human or non-human mammal (particularly a human). The medium containing the proteases (e.g. the wound) is contacted with a topical hydrogel composition comprising a hydrophilic polymer carrying multiple pendant sulphonyl groups, optionally with multiple pendant carboxylic groups, on each polymer molecule.

### FIELD OF THE INVENTION

The present invention relates to the modulation of protease enzymes, particularly but not exclusively in the treatment of skin lesions (wounds), particularly chronic (e.g. ulcerous) skin lesions and acute skin lesions at risk of, or showing signs of, becoming chronic, in humans and other mammals, particularly humans. More particularly, the invention relates to the use of a hydrogel composition or dressing for treatment of such wounds, for example chronic ulcerous skin lesions, to promote their healing.

The present invention develops the concept of “Pro-Ionic™” treatment of wounds introduced in our PCT patent application No. PCT/GB2006/002632 (publication no. WO/2007/007115), the contents of which are incorporated herein by reference, in which a hydrogel dressing in contact with the wound provides in use a controlled-moisture environment for the wound and selective uptake of proteins and ions from the wound, to stimulate and/or maintain the wound healing process.

More particularly, according to the present invention the hydrogel has surprisingly been found to modulate, for example activate or deactivate, protease enzymes, for example in skin lesions, particularly chronic ulcerous skin lesions.

Without wishing to be bound by theory, the hydrogel is believed to mimic the function of natural glycosminoglycans of a normal healing wound, and in particular certain sulphonated glycosaminoglycans of the extracellular matrix such as heparin, using a moist wound healing environment where the water levels are controlled to avoid the disadvantages of too much or too little moisture. In the case of chronic wounds, the hydrogel suppresses the proteolytic action of proteases that lead to chronic failure of the wound to heal and thus stimulates and/or maintains the normal healing process. In the case of acute wounds, the dressing suppresses a tendency towards chronic failure to heal, and stimulates and/or maintains the normal healing process.

The hydrogel used is a certain type of hydrous hydrophilic (ionic) polymer, described in more detail below. The ions covalently linked to the polymer molecule are generally anions; the cations are generally present as counterions (generally mono- or di-valent cations such as metal ions or primary or substituted ammonium ions). The hydrogel, including its associated water and ions, provides deactivation of proteases and one or more, for example simultaneously any two or more, of the following additional beneficial effects on the wound, without the need for other specific bioactive agents, namely: (1) beneficial antimicrobial action, (2) beneficial wound debridement, (3) beneficial skin conditioning, (4) reduction in wound odour, (5) beneficial pain relief, and (6) in combination, beneficial suppression of the processes which lead to, and/or maintain, a chronic wound with beneficial wound bed stimulation and/or maintenance of the healing process (see also Appendix A). Preferably, the said additional beneficial effects on the wound include beneficial antimicrobial action and simultaneously one or more, more preferably two or more, more preferably three or all, of effects (2) to (6). The said additional benefits may include the removal of microbes, e.g. bacteria, and simultaneously one or more, more preferably two or more, more preferably three or all, of effects (2) to (6).

### BACKGROUND OF THE INVENTION

### Lesion Healing Process

The normal process of healing of a skin lesion (wound) typically proceeds via four distinct sequential stages or phases, namely haemostasis, inflammation, proliferation and maturation.

Haemostasis is the vascular response stage, occurring immediately after the insult is suffered, and normally lasts for up to about three days in humans. The wound may bleed initially, and the blood then clots.

Inflammation normally arises about one after the insult, and typically continues until about six days after the insult. Inflammation involves one or more of redness, heat, swelling and pain. The wound starts to exude fluid, which serves to remove debris, and proteases are released into the wound area. White blood cells and macrophages begin to congregate in the lesion zone, the former to clear debris and the latter for phagocytosis and to release growth factors to stimulate fibroblasts. During this phase the extracellular matrix is constructed.

Proliferation normally arises about four days after the insult, and typically continues until about 21 days after the insult, and involves the gradual formation of granulation tissue to fill the lesion zone. The redness, heat, swelling and pain gradually subside during this phase. For these reasons, granulation and contracture are sometimes identified as sub-phases within the proliferation phase. During proliferation, the macrophages stimulate vascular endothelial growth factor (VEGF) to stimulate new blood vessel growth, and the concentration of fibroblasts increase, producing collagen for the new tissues.

The maturation phase normally arises about 21 days after the insult, and typically continues for several weeks, months or even years thereafter. Maturation involves contraction of the wound, growth of new epithelial tissue covering the wound, and possibly scar formation. During this phase myofibroblasts develop from the fibroblasts and the collagen fibres gradually mature and become relatively more organised.

Generally, different parts of a wound heal at different rates, so that it is common for some parts of a normal wound to be at a more advanced stage of healing than others.

The above timescale of a normal wound is provided for general illustration only, and is not definitive for all normal wound healing. The present invention is not limited by any requirement that the normal wound healing process must follow any particular pathway or timescale.

### Chronic Ulcerous Skin Lesions

Chronic skin lesions arise when a skin wound generally fails to follow an appropriate timely healing process to achieve the normal sustained and stable anatomic and functional integrity of the healed tissue. Generally speaking, a skin lesion which has failed to make at least substantial progress towards healing within a period of at least about three months, or which has become stable in a partially healed state for more than about three months, could be categorised as chronic, although even this general guide is not an absolute marker as the age and fitness of the patient, as well as other factors such as diseases or disorders suffered by the patient (for example, circulatory disorders), can significantly lengthen the normal healing process. A skin lesion which is unhealed after at least about one month, for example after at least about six months, can be categorised as chronic.

A chronic skin lesion is ulcerous where it involves focal loss of the epidermis and at least part of the dermis.

Malignant or pre-malignant chronic ulcerous skin lesions may arise in connection with a primary cancer of the skin, or with a metastasis to the skin from a local tumour or from a tumour in a distant site. They may be draining or non-draining. They may, for example, take the form of a cavity, an open area on the surface of the skin, skin nodules, or a nodular growth extending from the surface of the skin.

Benign chronic ulcerous skin lesions are not associated with cancer, and include venous leg ulcers, venous foot ulcers, arterial leg ulcers, arterial foot ulcers, decubitus ulcers (e.g. pressure sores, bedsores), post-surgical ulcerous lesions and chronic burn lesions. They may, for example, take the form of a cavity, an open area on the surface of the skin, skin nodules, or a nodular growth extending from the surface of the skin. Typically, they comprise an open granulating area on the surface of the skin.

Chronic ulcerous skin lesions are usually accompanied by other chronic symptoms apart from the failure of the normal healing process. Typical accompanying chronic symptoms include one or more of pain, exudation, malodour, excoriation, spreading of the wound, tissue necrosis, irritation and hyperkeratosis. Such symptoms can be extremely debilitating and embarrassing for patients, and can seriously harm the patient's quality of life. In severe cases, they can require amputation of limbs or even death.

Chronic ulcerous skin lesions can also be categorised according to their exudation. General categorisation is into the three categories “high exudation”, “medium exudation” and “low exudation”. Exudate management is a particularly difficult task for the caring professional attending to the patient. A balance needs to be struck between the desire to remove exudate to maintain the patient's quality of life at as high a level as possible, and maintenance of an appropriate level of fluid to prevent the lesion becoming too dry or too wet.

### The Role of Proteases in Wound Healing

Proteases are proteolytic enzymes that play an important role in the various stages of normal wound healing. During angiogenesis, for example, proteases are expressed at the growing tip of blood vessels to facilitate vascular invasion. Proteases also assist in debridement and cleansing of the wound, for example by removing necrotic tissue, foreign bodies and bacteria. During the reconstructive and remodelling phase, proteases digest the extracellular matrix and assist in tissue remodelling.

Numerous types of proteases have been identified as having roles in wound healing, for example, neutrophil-derived elastase, plasmin and matrix metalloproteins (MMPs). For further information, see for example Trengove et al, “Analysis of the Acute and Chronic Wound Environments: the Role of Proteases and their Inhibitors” in Wound Repair and Regeneration, Vol. 7, No. 6, November 1999, pp 442-452, the contents of which are incorporated herein by reference.

### Prior Art Treatments

An example of a prior art hydrogel wound dressing is Vigilon™ Primary Wound Dressing, available from Bard Medical. The hydrogel sheet comprises insoluble cross-linked polyethylene oxide as hydrophilic polymer and the manufacturer's information is that it contains about 96 wt % water.

WO-A-00/07638, the contents of which are incorporated herein by reference, discloses bioadhesive hydrogel compositions and their use in wound dressings. The polymer composition is stated to preferably comprise also a non-hydrophilic (hydrophobic) polymer, and may comprise a specifically antimicrobial agent such as citric acid or stannous chloride. No information is given as to any effects of the hydrogel compositions on the proteases of wounds, for example human skin wounds. More generally, there is no teaching that the polymer per se in the hydrogel, including its associated water and ions, provides any protease inhibition alone or in combination with the additional beneficial effects on the wound mentioned as (1) to (5) above, without the need for other bioactive agents.

It is known to apply dressings to chronic skin lesions, with the aim of promoting their healing. Examples of such prior art dressings for chronic ulcerous skin lesions include Aquacel™ (ConvaTec) (http://www.dressings.org/Dressings/aquacel.html), Intrasite™ (Smith & Nephew) (http://www.dressings.org/Dressings/intrasit.gel.html) and Avance™ (Medlock Medical) (http://www.medlockmedical.com/woundcare/avance.htm).

Generally speaking, and without commenting specifically on the particular examples given above, prior art dressings for chronic ulcerous skin lesions suffer from a variety of problems. For example, they can cause maceration of peri-wound areas, they can absorb wound exudate only partially, they can cause contact dermatitis, varicose eczema or skin stripping (e.g. due to aggressive or allergenic adhesive materials). Furthermore, even in cases where the prior art dressings for chronic skin lesions contribute to successful healing, scarring of the healed wound and poor quality of healed tissue can often be found.

The prior art dressings for chronic ulcerous skin lesions can also be slow and difficult to apply and change, and require frequent changing. Many patients experience considerable—sometimes unbearable—pain associated with changing of the dressing, over and above the often considerable general pain associated with the lesion itself. The use of opiate painkillers to deal with this pain can lead to opiate dependency and addiction.

Prior art dressings that require frequent changing cause a significant increase in costs to healthcare services and providers, as a nurse or other healthcare professional needs to attend the patient correspondingly more often. In addition, the material costs of the dressings clearly are higher because of the frequent application of fresh dressings.

The Trengrove et al publication cited above proposed that protease inhibitors could be used in the treatment of chronic wounds in conjunction with any treatments using growth factors. However, protease inhibitors are relatively expensive speciality chemicals and their addition to normal or normalising wounds can do more harm than good. In addition, they do not overcome the problem of pain and the other problems of the dressings themselves.

In an article entitled “A small study in healing rates and symptom control using a new sheet hydrogel dressing” in Journal of Wound Care, Jul. 2004, 13(7), and in a poster presentation at the Tissue Viability Society (TVS) Conference in Torquay, UK, in April 2003, available on http://www.activahealthcare.co.uk/pdf/cs-actiformcool2.pdf, the contents of all of which are incorporated herein by reference, Sylvie Hampton described a study into the effects of a sheet hydrogel dressing on chronic leg and foot ulcers of at least six months duration (average 9 months to two years) in 16 human patients. The pre-treatment ulcers of almost all of the patients were either high exudation or medium exudation. The sheet hydrogel dressing was supplied by Activa Healthcare of Burton-upon-Trent, UK (tel: +44 8450 606 707; web: www.activahealthcare.co.uk) under the name ActiFormCool™.

The results published by Sylvie Hampton showed the potential for substantial advantages deriving from the use of ActiFormCool™ as a dressing in the treatment of chronic leg and foot ulcers. However, neither the Journal of Wound Care article nor the poster presentation mentioned above disclosed the underlying nature of the therapeutic effect or the nature of any active component of the composition of ActiFormCool™. No information was given as to any effects of the hydrogel compositions on the protease levels of wounds. More generally, there was no teaching that the polymer per se in the hydrogel, including its associated water and ions, provides any protease modulation of activity (enhancement or inhibition) alone or in combination with the beneficial effects on the wound mentioned as (1) to (5) above, without the need for other bioactive agents.

In the following description, the expressions “modulation”, “modulator”, “modulate” and related expressions shall be considered as equivalent to and interchangeable with “enhancement or inhibition”, “enhancer or inhibitor”, “enhance or inhibit” and related expressions.

### Basis of the Present Invention

The present invention is based on our surprising finding that the hydrogels described below exhibit modulation of protease activity and can therefore function as protease modulators, for example in a wound dressing, particularly a wound dressing for a chronic (e.g. ulcerous) wound. Furthermore, we have found that in use the dressing is a self-regulating system, whereby the extent of protease modulation can reduce as the concentration of proteases in the wound reduces and as the wound approaches a normalised state, so that undesirable levels of protease modulation are not found in practice. Furthermore, this self-regulation is exhibited by fresh dressings newly applied in dressing-changes, so that it appears that the hydrogel system responds sensitively to the state of healing of the wound.

We have also surprisingly found that particular embodiments of the hydrogels, as described below, provide a particularly substantial effect in enhancing the protease modulation, particularly inhibition for some proteases, thus leading to faster healing with potentially a better outcome in terms of texture, strength and colour of the healed wound.

Broadly speaking, the hydrogels for use in the present invention have multiple pendant sulphonyl groups, and optionally also multiple pendant carboxylic groups, on each polymer molecule of the hydrogel.

As described in more detail below, the hydrogel may comprise a polymer which includes, but is not limited to, homopolymers, copolymers and all mixtures and combinations thereof. The monomers as described herein may suitably be used in admixture with each other or with other monomers. In one particularly useful embodiment of the invention, a monomer which has a first countercation associated with it may be used in admixture with one or more monomer which has/have one or more second/further countercation(s) associated with it/them. The monomers in their anionic form (i.e. disregarding the counter-cation) may be the same or different.

By “pendant sulphonyl groups” we mean sulphonyl (—SO₂—) containing groups, most particularly sulpho (—SO₂—OH) groups in acid or salt form or organic groups which include sulpho (—SO₂—OH) groups in acid or salt form, which extend from the carbon atom containing chain (“carbon chain”) of the polymer molecule and are covalently linked (pendant) to the carbon chain. Where the sulphonyl containing group is an organic group which includes the sulphonyl moiety, e.g. in a sulpho (—SO₂—OH) group in acid or salt form, the sulphonyl moiety is preferably located at or near the terminal free end of the organic group, i.e. the end distant from the carbon chain of the polymer molecule.

Some or all of the sulpho groups (—SO₂—OH) groups in acid or salt form may, if desired, be O-linked to the carbon chain of the polymer molecule, for example as organic sulphate groups.

Where sulpho groups or some of them are present in salt form, the salt form may suitably be an alkali or alkaline earth or other multivalent (e.g. transition) metal or ammonium or organo-ammonium salt of the acid form (—SO₂—OH). For example, the salt form may be the sodium, potassium, lithium, caesium, calcium, magnesium, zinc or ammonium salt or combinations thereof. Preferably the salt form will comprise sodium ions, in combination with one or more other salt forms such as, for example, potassium or ammonium. A combination of sodium and potassium counterions can be particularly suitable.

The organic sulphonyl containing groups or some of them may contain a carboxylate or carboxamido linkage unit. The polarity of these species, in conjunction with the sulphonyl groups, seems to play a part in achieving the desirable effects underlying the present invention. It is preferred that the carboxylate or carboxamido linkage unit, when present, is closer to the carbon chain of the polymer than the sulphonyl moiety.

By “pendant carboxylic groups” we mean carboxylate (—CO₂—) containing groups, most particularly carboxylic acid (—CO₂H) groups in acid or salt form or organic groups which include carboxylic acid (—CO₂H) groups in acid or salt form, which extend from the carbon atom containing chain (“carbon chain”) of the polymer molecule and are covalently linked (pendant) to the carbon chain. Where the carboxylate containing group is an organic group which includes the carboxylate moiety, the carboxylate moiety is preferably located at or near the terminal free end of the organic group, i.e. the end distant from the carbon chain of the polymer molecule.

Where carboxylic acid groups or some of them are present in salt form, the salt form may suitably be an alkali or alkaline earth or other multivalent (e.g. transition) metal or ammonium or organo-ammonium salt of the acid form (—CO₂H). For example, the salt form may be the sodium, potassium, or ammonium salt or combinations thereof. Preferably the salt form will comprise sodium ions, in combination with one or more other salt forms such as, for example, potassium, or ammonium. A combination of sodium and potassium counterions can be particularly suitable. Where a combination of counterions is present in the hydrogel, any multivalent counterion (e.g. one or more of magnesium, zinc, calcium) is suitably present in a total molar proportion of up to about 5 mol % relative to the univalent (e.g. sodium) ions.

We have found that the hydrogels can be particularly effective as protease modulators when at least some of the sulphonyl and, if present, carboxylic, groups are present in salt form and the nature and/or relative number of associated countercations are selected as described in more detail below.

The finding, for the first time in these hydrogels, of an intrinsic protease modulatory action makes effective treatment available to a wider class of patients having a range of wound conditions, including chronic ulcerous skin lesions and in particular chronic leg and foot ulcers that are refractory to prior art treatments. Patients who have adverse reactions to specific protease modulator chemicals, e.g. protease inhibitor chemicals, or for whom the administration of specific protease modulators might risk side effects or other disadvantages, will now benefit from the present invention. The present invention assists in bringing the potential advantages of protease modulation in wound care to the general public with reduced risk of adverse effects. In addition, since the hydrogels used in the present invention also have antimicrobial and other beneficial effects as noted above, the dressings are potentially of great benefit to patients who have reactions to certain classes of antibiotics, painkillers or other bioactive agents conventionally used in, or in conjunction with, wound dressings, or who are addicted to or dependent on opiate or other powerful painkillers conventionally used in conjunction with wound care. Those people will be treatable using the present invention—in which the use of other bioactive agents such as specific protease modulators, antibiotics or painkillers can be avoided—whereas previous treatment protocols were restricted by the need to avoid the problematic chemical agents such as antibiotics, painkillers or other bioactive agents. Therefore, the novel findings constitute and make available a novel therapeutic application.

Sulphonated hydrophilic polymers are known to have antagonist activity towards fibroblast growth factor-2 (FGF-2), and consequently their use as potential inhibitors of FGF-2-induced endothelial cell proliferation in angiogenesis and tumour growth has been proposed (S Liekens et al, Molecular Pharmacology, 56, pages 204 to 213 (1999)). In view of this, our novel finding that the polymers can promote healing of wounds, when applied as a hydrous hydrophilic ionic hydrogel in contact with a wound, through protease modulation either alone or in combination with one or more of the beneficial effects on the wound mentioned as (1) to (5) above, without the need for other bioactive agents, is surprising and not obvious. Our current understanding of the mode of action of the invention is explained below, and is compatible with the reported FGF-2-antagonistic activity of the (un-crosslinked) polymers in solution.

### BRIEF DESCRIPTION OF THE INVENTION

According to a first aspect of the present invention, there is provided a method of modulating proteases, comprising contacting a medium containing the proteases for an effective period of time with a hydrogel composition comprising a hydrophilic polymer carrying multiple pendant sulphonyl groups, optionally with multiple pendant carboxylic groups, on each polymer molecule. The method may be a method of inhibiting proteases comprising contacting a medium containing the proteases for an effective period of time with a hydrogel composition comprising a hydrophilic polymer carrying multiple pendant sulphonyl groups, optionally with multiple pendant carboxylic groups, on each polymer molecule.

The method may suitably be used in the treatment of a wound, for example, a chronic ulcerous skin lesion, in a human or non-human mammal, particularly a human, in which case the medium containing the proteases is the wound tissue or fluid. For this use, the hydrogel composition is preferably provided as a topical composition, for example in a wound dressing.

According to a second aspect of the present invention, there is provided a method of modulating proteases in a wound, for example a chronic ulcerous skin lesion, in a human or non-human mammal, particularly a human, comprising contacting the wound for an effective period of time with a topical hydrogel composition comprising a hydrophilic polymer carrying multiple pendant sulphonyl groups, optionally with multiple pendant carboxylic groups, on each polymer molecule

At least some of the pendant groups are preferably present in salt form, so that charge-balancing countercations other than H⁺ are present in the hydrogel associated with the pendant groups. It is particularly preferred that two or more different countercations will be present in the hydrogel, and most preferably these are selected from sodium, potassium, ammonium or organo-ammonium cations (primary ammonium, secondary ammonium, tertiary ammonium and quaternary ammonium cations).

It is preferred that any two or more different countercations associated with pendant anionic groups of the hydrogel, are provided in a controlled relative molar proportion according to the nature and extent of hydration of the countercations (e.g. according to the respective kosmotropic and chaotropic nature of the anions and cations or the position of the countercations in the Hofmeister series of cations).

The polymers (including copolymers), both crosslinked and non-crosslinked, of the invention preferably comprise pendant anionic groups that are kosmotropic (water order makers) in nature. The cationic counterion is preferably chaotropic (disorder maker) or, at most, weakly kosmotropic. The polymers of the invention may contain a mixture of pendant anionic groups possessing different degrees of water order making e.g. varying in kosmotropic strength, for example comprising phosphate, phosphonate, sulphate, sulphonate and carboxylate and combinations there of. The extent of kosmotropic and chaotropic behaviour has been quantified by thermodynamic parameters such as the Jones Dole B viscosity coefficients. Preferred values of the Jones Dole B coefficient for the anion kosmotropic behaviour are larger than 0.1 and preferably larger than 0.2. Preferred values of the Jones Dole B coefficient for the cation chaotropic behaviour are larger than −0.1.

The polymers of the invention may thus contain a mixture of chaotropic and kosmotropic ions. The molar ratio of chaotropic to kosmotropic cation is preferably less than about 500:1, for example less than about 250:1, preferably less than about 200:1, for example less than about 100:1, for example less than about 80:1, for example less than about 50:1, and preferably more than about 2:1. For example, the ratio may be between about 2:1 and about 250:1, for example between about 5:1 and about 200:1, for example between about 5:1 and about 100:1, for example between about 7:1 and about 100:1, for example between about 10:1 and about 100:1.

The polymers of the invention may also comprise combinations of pendant anionic group differing in the extent of the kosmotropic behaviour. The molar ratio of pendant anionic kosmotropic groups with relatively larger Jones Dole B viscosity coefficients (higher kosmotropic behaviour) to pendant anionic kosmotropic groups with relatively smaller Jones Dole B viscosity coefficients (lower kosmotropic behaviour) is preferably between about 1000:1 and about 1:1000, more preferably between about 200:1 and about 1:200, and even more preferably between about 100:1 and about 1:100.

Thus, if the countercations are identified as the first and second countercations such that the first is the relatively more strongly hydrated according to the Hofmeister series of cations and the second is the relatively more weakly hydrated according to the Hofmeister series of cations, then it is preferred according to the present invention that the molar ratio of the first to the second countercations in the hydrophilic polymer is less than about 250:1, preferably less than about 200:1, for example less than about 100:1, for example less than about 80:1, for example less than about 50:1, and preferably more than about 2:1. For example, the ratio may be between about 2:1 and about 250:1, for example between about 5:1 and about 200:1, for example between about 5:1 and about 100:1, for example between about 7:1 and about 100:1, for example between about 10:1 and about 100:1.

The first cation may, for example, be sodium and the second may, for example, be selected from potassium, primary ammonium, secondary ammonium, tertiary ammonium and quaternary ammonium, or the first may be potassium and the second may be selected from primary ammonium, secondary ammonium, tertiary ammonium and quaternary ammonium

In one particular embodiment, the hydrophilic polymer is a homopolymer or copolymer comprising polymerised (co)monomer(s) carrying groups which provide the pendant groups of the polymer. One or more additional monomer may optionally be present in the polymer if desired, provided that the ionic balance of the polymer mentioned above is maintained. At least some of the said pendant groups of the polymer are in salt form with a first countercation and a second countercation, different from the first. The said countercations are selected from relatively weakly hydrated cations according to the Hofmeister series of cations, for example sodium, potassium, primary ammonium, secondary ammonium, tertiary ammonium and quaternary ammonium cations. The countercations are preferably chosen such that the first is the relatively more strongly hydrated according to the Hofmeister series of cations and the second is the relatively more weakly hydrated according to the Hofmeister series of cations. For example, the first cation may be sodium and the second may be selected from potassium, primary ammonium, secondary ammonium, tertiary ammonium and quaternary ammonium, or the first may be potassium and the second may be selected from primary ammonium, secondary ammonium, tertiary ammonium and quaternary ammonium

The molar ratio of the said first to the second countercations in the hydrophilic polymer is preferably less than about 250:1, preferably less than about 200:1, for example less than about 100:1, for example less than about 80:1, for example less than about 50:1, and preferably more than about 2:1. For example, the ratio may be between about 2:1 and about 250:1, for example between about 5:1 and about 200:1, for example between about 5:1 and about 100:1, for example between about 7:1 and about 100:1, for example between about 10:1 and about 100:1.

The polymer may suitably be formed by polymerisation of monomers in which the groups which provide the pendant groups of the polymer are in salt form, such that the molar ratio of the monomer(s) in which the salt cation is the said first countercation in the hydrophilic polymer, to the monomer(s) in which the salt cation is the said second countercation in the hydrophilic polymer, is, correspondingly, preferably less than about 250:1, preferably less than about 200:1, for example less than about 100:1, for example less than about 80:1, for example less than about 50:1, and preferably more than about 2:1. For example, the ratio may be between about 2:1 and about 250:1, for example between about 5:1 and about 200:1, for example between about 5:1 and about 100:1, for example between about 7:1 and about 100:1, for example between about 10:1 and about 100:1. These ratios relate to univalent molar equivalents; in the case of multivalent cations associated with the (univalent) anionic groups of the monomer(s), the molar amounts of the monomer(s) will be correspondingly adjusted.

The hydrogel composition comprises a polymer matrix holding a liquid (normally aqueous) phase retained within the hydrogel. The polymer matrix may for example be cross-linked or entangled, preferably cross-linked. The degree of cross-linking may be varied as desired. The polymeric matrix preferably consists of a cross-linked hydrophilic polymer. The liquid phase may, if desired, incorporate one or more other bioactive agents (e.g. particularly agents soluble or miscible in the liquid held within the polymer matrix of the hydrogel) to assist the healing process of the chronic skin lesion, or may be free or substantially free of such bioactive agents. It is a preferred feature of the present invention, however, that the hydrogel composition per se can be effective for the protease modulation, without the need for other bioactive agents. Therefore, in one embodiment of the present invention the hydrogel composition is substantially or entirely free of added bioactive agents having specific therapeutic or other physiological activity.

The hydrogel composition is preferably used in sheet form. The hydrogel composition is preferably prepared in sheet form by polymerisation of a laid down layer of a liquid pre-gel mixture of polymerisable components, which are then cured to provide the polymerised mass. Preferably all or substantially all of the desired components of the hydrogel composition, including any water, are present in the pre-gel, and that no or substantially no drying or other adjustments are required after polymerisation (apart from minor conventional conditioning).

The contacting of the protease containing medium of a wound with the hydrogel composition comprising a hydrophilic polymer carrying multiple pendant sulphonyl groups, optionally with multiple pendant carboxylic groups, on each polymer molecule preferably takes place for a period of time or for a sequence of time periods to promote healing in addition to the protease modulation, preferably with simultaneous reduction in one or more of pain, exudation, malodour, excoriation, spreading of the wound, tissue necrosis, irritation and hyperkeratosis.

The effective amount of pendant sulphonyl groups, optionally with multiple pendant carboxylic groups, and the use of countercations for the salt forms thereof, including selection of the nature and/or molar ratio of any said two or more countercations present, for treating the wound, will vary from subject to subject, but generally speaking the effective amount is as described in more detail below, in the section headed “Detailed Description of the Invention; The Hydrogel, Dressing and Treatment”. Adjustments to the sulphonyl and optional carboxylic groups and/or the nature and/or molar ratio of any said two or more countercations present to suit individual subjects will be within the capacity of one skilled in the art, following simple experimental procedures.

The hydrophilic polymer used in the present invention may, if desired, comprise further multiple pendant anionic groups, in addition to the sulphonyl groups and optional carboxylic groups present. Where such additional anionic groups are present, they will typically be in relatively small numbers in comparison with the sulphonyl and optional carboxylic groups. Any such additional anionic groups may be present in acid or salt form, provided that the ionic balance of the polymer mentioned herein is maintained. Examples of such additional pendant anionic groups that may be present are relatively strongly hydrated anions according to the Hofmeister series of anions, for example phosphate or phosphonyl groups.

The effective period of time will vary from subject to subject, but generally speaking an effective period of time will be up to about six weeks, for example between about 3 days and 6 weeks, depending on the seriousness of the wound and whether it is acute or chronic. Regular changes of the dressing will be required, particularly with more serious and exuding wounds. The time between changes of dressing will generally be in the range of about 2 to about 7 days, preferably about 3 to about 7 days. The hydrogel composition used in the present invention seems to require fewer changes per week on average, than prior conventional dressings used for the treatment of chronic ulcerous skin lesions. For example, a study of 20 patients having chronic leg and foot ulcers showed that the prior art dressings required on average 3.00 changes per week, whereas the dressing according to the present invention required on average 1.75 changes per week. This is highly advantageous, both in terms of cost and manpower demands on health services and in terms of the pain and inconvenience to patients.

According to a third aspect of the present invention, there is provided a hydrogel composition comprising a hydrophilic homopolymer or copolymer carrying multiple pendant sulphonyl groups, optionally with multiple pendant carboxylic groups. The polymer comprises polymerised (co)monomer(s) each carrying groups which provide the pendant groups of the polymer. One or more additional co-monomer may optionally be present in the polymer if desired, provided that the ionic balance of the polymer mentioned below is maintained. At least some of the said pendant groups of the polymer are in salt form with a first countercation and a second countercation, different from the first. The said countercations are selected from relatively weakly hydrated cations according to the Hofmeister series of cations, for example sodium, potassium, primary ammonium, secondary ammonium, tertiary ammonium and quaternary ammonium cations. The molar ratio of the said first and second countercations in the hydrophilic copolymer is less than about 250:1, preferably less than about 200:1, for example less than about 100:1, for example less than, about 80:1, for example less than about 50:1, and preferably more than about 2:1, for example, between about 2:1 and about 250:1, for example between about 5:1 and about 200:1, for example between about 5:1 and about 100:1, for example between about 7:1 and about 100:1, for example between about 10:1 and about 100:1, whereby the first is the relatively more strongly hydrated according to the Hofmeister series of cations and the second is the relatively more weakly hydrated according to the Hofmeister series of cations. For example, the first cation may be sodium and the second may be selected from potassium, primary ammonium, secondary ammonium, tertiary ammonium and quaternary ammonium, or the first may be potassium and the second may be selected from primary ammonium, secondary ammonium, tertiary ammonium and quaternary ammonium. The further details of the hydrogels according to the first and second aspects of the present invention, described herein, apply equally to the third aspect of the invention.

The hydrogel composition according to the third aspect of the present invention may be for use in the treatment of a wound, for example a chronic skin lesion, in a human or non-human mammal, particularly a human.

According to a fourth aspect of the present invention, there is provided a hydrogel composition for use as a protease modulator, particularly in the topical treatment of a wound, for example a chronic skin lesion, in a human or non-human mammal, particularly a human, the hydrogel composition comprising a hydrophilic polymer carrying multiple pendant sulphonyl groups, optionally with multiple pendant carboxylic groups. At least some of the said groups may suitably be in salt form so that the hydrophilic polymer comprises two or more countercations. The further details of the hydrogels according to the first and second aspects of the present invention, described herein, apply equally to the fourth aspect of the invention. The hydrogel composition according to the third aspect of the present invention constitutes a preferred embodiment of the fourth aspect. For use as a topical wound treatment, the hydrogel composition is preferably provided in a wound dressing.

According to a fifth aspect of the present invention, there is provided the use of a hydrogel composition comprising a hydrophilic polymer carrying multiple pendant sulphonyl groups, optionally with multiple pendant carboxylic groups, in the preparation of a topical medicament for use as a protease modulator in vivo, particularly in the treatment of a wound, for example a chronic skin lesion, in a human or non-human mammal, particularly a human. At least some of the said groups may suitably be in salt form so that the hydrophilic polymer comprises two or more countercations. The further details of the hydrogels according to the first and second aspects of the present invention, described herein, apply equally to the fifth aspect of the invention. The hydrogel composition according to the third aspect of the present invention constitutes a preferred embodiment of the hydrogel composition used in the fifth aspect.

A wound to be treated using any of the first to fifth aspects of the present invention may be of any type, acute or chronic. The wound may for example be a chronic ulcerous skin lesion, for example a malignant or pre-malignant chronic ulcerous skin lesion or a benign chronic ulcerous skin lesion.

The chronic ulcerous skin lesion may particularly be selected from venous leg ulcers, venous foot ulcers, arterial leg ulcers, arterial foot ulcers, decubitus ulcers (e.g. pressure sores, bedsores), post-surgical ulcerous lesions and chronic burn lesions.

The chronic ulcerous skin lesion may be a high exudation lesion, a medium exudation lesion or a low exudation lesion.

The hydrogel composition has the capacity to absorb many times (e.g. at least about 2.5 times, for example at least about 5 times, for example at least about 10 times, for example between about 10 and about 50 times) its own weight of exudate or other fluid in 24 hours. Therefore, the exudate management capacity of the composition can be selected according to the intended target patients and lesions for treatment. The hydrogel preferably has a water activity greater than 0.4, for example greater than 0.5, for example greater than 0.6, for example greater than 0.7, preferably greater than 0.8, preferably greater than 0.9, preferably greater than 0.95, preferably greater than 0.97 but less than 0.99 in the absence of maceration. In the presence of maceration the hydrogel preferably has a water activity less than 0.95, more preferably less than 0.9. As mentioned below, in some instances the water activity of the hydrogel may be substantially lower than 0.4. As described in more detail below, one particularly suitable hydrogel for use in the present invention may have a water activity in the range of 0.6 to 0.89.

The present invention has been found to provide, through the protease modulation, a wound healing and/or microbial kill effect in the absence of other protease modulators, antimicrobial agents (e.g. antibiotics) and/or painkilling agents.

Therefore, the aspects of the present invention as defined herein are suitably provided for use on subjects who, at the start of their treatment according to the present invention, are not receiving (and preferably also who have not been receiving recently, i.e. in the previous time period of about 2 weeks) other, separately administered, protease modulators, antimicrobial and/or painkilling agents, and more preferably still for use on subjects who, at the start of their treatment according to the present invention, are not receiving other protease modulators, antimicrobial and/or painkilling agents, whether separately administered or incorporated in the hydrogel.

Such other agents are typically so-called “small-molecule” (non-polymeric, non-protein) protease modulators, antimicrobial and/or painkilling agents (for example, having molecular weights less than about 1000). Such antimicrobial agents include antibiotics, such as for example antibiotics of the penicillin, cephalosporin, macrolide, aminoglycoside and teracycline families and combinations thereof. Such painkilling agents include analgesics of the narcotic and non-narcotic families and combinations thereof, such as for example nitrous oxide (Entonox), salicylates such as aspirin, acetaminophen, nonsteroidal anit-inflammatory drugs such as ibuprofen, opiates and opioids such as codeine, propoxyphene (e.g. Darvon and Wygesic), meperidine (Demerol) and morphine, acetaminophen/codeine (e.g. Tylenol with Codeine and Tylox), aspirin/codeine (e.g. Empirin with Codeine), propoxyphene/aspirin (e.g., Darvon Compound-65); and aspirin/caffeine/butalbital (Florinal).

Apart from immediately apparent cost advantages in avoiding the use of other protease modulators, antimicrobial and/or painkilling agents in the treatments, the present invention makes available new therapeutic applications by avoiding over-prescription of protease inhibitors, antibiotics (thereby reducing the risk of emergence of antibiotic-resistant strains or populations of bacteria), and opens effective wound treatments to subjects who are, or might be, sensitive, reactive or allergic to certain classes of protease modulators, antibiotics, painkillers or other bioactive agents, or who are addicted to or dependent on opiate or other painkillers (analgesics) conventionally used in conjunction with wound care (or who are actually or potentially susceptible to such addiction or dependence). See Appendix A for further information as to the range of effects of the hydrogels.

Furthermore, the application of the present invention to subjects who are, at the start of their treatment according to the present invention, not receiving (or have not been recently receiving) other, separately administered, protease modulators, antimicrobial and/or painkilling agents, is technically advantageous in that such patients have no psychological reliance on the protease modulators, antimicrobial and/or painkilling agents and therefore are psychologically receptive to the simpler treatment according to the present invention. The psychological receptiveness of a patient to the treatment about to be delivered can be an important factor in improving the clinical outcome for the patient, and can provide an unexpected and unquantifiable advantage in the treatment.

A similar psychological reliance can be observed in patients who are, at the start of treatment according to the present invention, receiving (or have recently been receiving) one or more other, different, hydrogel or hydrocolloid treatment for the same purpose (i.e. for the same wound). Therefore, the aspects of the present invention as defined herein are suitably provided for use on subjects who, at the start of their treatment according to the present invention, are not receiving (and preferably also who have not been receiving recently, i.e. in the previous time period of about 2 weeks) one or more other, different, hydrogel or hydrocolloid treatment for the same purpose.

It is well known that water in hydrogels can be present in at least two forms, freezing and non-freezing, as measured by differential scanning calorimetry. In many examples of commercially available hydrogels the water is present only as non-freezing water. It has been found, however, that compositions with useful adhesive properties can be made which have both freezing and non-freezing water, and the water activity in such gels is generally high.

As discussed in more detail below, the beneficial effects of the hydrogel according to the present invention are believed to derive from the multiple pendant sulphonyl groups, optionally with multiple pendant carboxylic groups, of the polymer molecules. It is believed that these act in situ at the zone of contact with the wound to modulate protease activity, optionally with other effects such as selectively concentrating one or more naturally exuded salts in the ulcerous region of the lesion (the “wound bed”) and/or selectively absorbing one or more naturally exuded salts in the wound bed (see our PCT patent application No. PCT/GB2006/002632, publication no: WO/2007/007115). The hydrogel thus acts without the need for externally applied salt or other ionic aqueous solutions, and preferably also in the absence of salt or other ionic aqueous solutions in the liquid held within the polymer matrix of the hydrogel, so that the blocking mechanism preventing completion of the normal wound healing process is overridden, bypassed, shut off or otherwise disabled, and continuation of the normal wound healing process to substantial completion is enabled or initiated.

The selectivity of the protease modulation, particularly inhibition, is preferably achieved through the control of the counterion(s), if any, present on the sulphonyl groups or present on the multiple sulphonyl and carboxylic groups. Generally speaking, it is believed that selection of, say, sodium counterions on —SO₃⁻ groups (i.e. a sulpho group in salt form) will favour concentration of sodium salts (e.g. sodium chloride) in the wound bed, whereas selection of, say, potassium counterions on —SO₃⁻ groups will favour concentration of potassium salts (e.g. potassium chloride) in the wound bed whereas selection of, say, calcium counterions on —SO₃⁻ groups will favour concentration of calcium salts (e.g. calcium chloride) in the wound bed. For example, we believe that it will be advantageous for the molar ratio of sodium ions to potassium ions associated in the hydrogel composition (or sodium ions to other more weakly hydrated cations according to the Hofmeister series of cations) to be less than about 250:1, preferably less than about 200:1, for example less than about 100:1, for example less than about 80:1, for example less than about 50:1, and preferably more than about 2:1, for example, between about 2:1 and about 250:1, for example between about 5:1 and about 200:1, for example between about 5:1 and about 100:1, for example between about 7:1 and about 100:1, for example between about 10:1 and about 100:1. Other counterions may also be used, as discussed above, in which case the molar ratios stated above apply instead to first and second cations in place of sodium and potassium ions, the first being the relatively more strongly hydrated according to the Hofmeister series of cations and the second being the relatively more weakly hydrated according to the Hofmeister series of cations.

From this, it is now possible to control the healing process in wounds, for example in chronic ulcerous skin lesions, for the first time, without the need for externally applied salts or other bioactive agents apart from the dressing itself, and more particularly without the need for salts or other bioactive agents in the dressing apart from the hydrogel polymer matrix (including the associated water and the ions of the hydrogel polymer) of the dressing itself.

The protease modulation according to the present invention makes available simultaneous reduction of one or more undesirable characteristics of a chronic skin lesion selected from pain associated with the wound, pain associated with changing of the dressing, exudation, malodour, irritation and hyperkeratosis (see our PCT patent application No. PCT/GB2006/002632; publication no. WO/2007/007115).

As described in WO/2007/007115, undesirable effects of conventional dressings for chronic skin lesions, for example maceration, incomplete absorption of exudate, excoriation, scarring of the final healed tissue, contact dermatitis, varicose eczema or skin stripping can be reduced using the present invention.

As also described in WO/2007/007115, the dressing used in the present invention is easy to apply and change, with resultant cost savings and efficiency enhancements. Moreover, the number of dressing changes required is reduced substantially.

Unless specifically stated otherwise, or implicitly otherwise by the context, the examples and preferences expressed herein in relation to any one aspect of the invention apply equally to all the other aspects of the invention, both independently of each other or in any combination.

### DETAILED DESCRIPTION OF THE INVENTION

### The Hydrogel, Dressing and Treatment

The expression “hydrogel” and like expressions, used herein, are not to be considered as limited to gels which contain water, but extend generally to all hydrophilic gels, including those containing organic non-polymeric components in the absence of water. The gel forming agent may, for example, be selected from natural hydrophilic polymers, synthetic hydrophilic polymers, gelling hydrophilic biopolymers and all combinations thereof. The term “hydrogel” is used herein regardless of the state of hydration, and therefore includes, for example, hydrogels that are in a dehydrated or anhydrous state or in a state of partial hydration.

Hydrogels are described in greater detail in Hydrogels, Kirk-Othmer Encyclopedia of Chemical Technology, 4^{th} Edition, vol. 7, pp. 783-807, John Wiley and Sons, New York, the contents of which are incorporated herein by reference.

The expression “polymer” and like expressions, used herein, includes homopolymers, copolymers and all mixtures and combinations thereof. The expression “polymer” and like expressions, used herein, includes cross-linked and uncrosslinked polymers, as well as polymers characterised by entangled polymer chains. The expression “polymer” and like expressions, used herein, includes bicontinuous and higher multicontinuous intermeshing polymer systems, in which two or more polymers form identifiable intermeshing phases extending within the hydrogel mass.

Hydrogels are, generally speaking, hydrophilic polymers characterized by their hydrophilicity (i.e. capacity to absorb large amounts of fluid such as wound exudate) and insolubility in water: i.e. they are capable of swelling in water while generally preserving their shape.

The hydrophilicity is generally due to groups such as hydroxyl, carboxy, carboxamido, and esters, among others. On contact with water, the hydrogel assumes a swollen hydrated state that results from a balance between the dispersing forces acting on hydrated chains and cohesive forces that do not prevent the penetration of water into the polymer network. The cohesive forces are most often the result of crosslinking, but may result from electrostatic, hydrophobic or dipole-dipole interactions.

The hydrogels in the present invention include as a necessary component a hydrophilic polymer carrying multiple pendant sulphonyl groups on each polymer molecule, preferably in salt form counterbalanced by one or more cations.

Generally, the degree of sulphonylation of such a polymer is on average (number average) at least about one pendant sulphonyl group per linear 30 carbon atoms of the carbon atom backbone of the polymer, at least about one pendant sulphonyl group per linear 12 carbon atoms of the carbon atom backbone of the polymer, for example at least about one pendant sulphonyl group per linear six carbon atoms of the carbon atom backbone of the polymer. More preferably, the polymer will contain on average at least about two pendant sulphonyl groups per linear six carbon atoms of the carbon atom backbone of the polymer, for example up to about three pendant sulphonyl groups per linear six carbon atoms of the carbon atom backbone of the polymer. At the higher levels of sulphonylation it is preferred that pendant carboxylate groups will be substantially absent.

Most preferably, the polymer contains one pendant sulphonyl group per linear two carbon atoms of the carbon atom backbone of the polymer. Such a polymer is readily prepared by polymerising (meth)acrylic acid derivatives such as esters or amides using monomers containing one sulphonyl group per molecule. The sulphonyl groups may be present in acid, ester, salt or other suitable form, and may be covalently linked to the carbon atom backbone of the polymer. A suitable sulphonyl moiety is the —SO₃⁻ species, either in acid form (—SO₃H) or in salt form (—SO₃M, where M is a univalent metal counterion, or —SO₃MO₃S— where M is a divalent metal counterion), or the organic sulphate species (for example, —O—SO₃H in acid form, or in corresponding salt form). Suitable linking moieties include alkylene bridges, alkylene-ester bridges, —O— bridges and alkylene-amide bridges. The alkylene moieties may be straight or branched, saturated and preferably contain from 1 to about 8 carbon atoms.

Such hydrophilic polymers include, for example, polymers derived from (meth)acryloyloxyalkylsulphonates, polymers of sulpho-substituted acrylamides such as acrylamidoalkanesulphonic acids, polymers of salts of any of the foregoing (for example, alkali or alkaline earth metal salts or ammonium or quaternary organ-ammonium salts), or any combination thereof. Mixtures of such polymers with each other are also envisaged. Such polymers may, if desired, be used together with sulpho-free polymers. Such other polymers, if present, may suitably be selected from homopolymers or copolymers of acrylic and methacrylic acid esters, including hydroxyalkyl (meth)acrylates, 2-(N,N-dimethylamino)ethyl methacrylate, polymers and copolymers of other substituted and unsubstituted acrylamides, polymers and copolymers of N-vinylpyrrolidinone, and polyelectrolyte complexes.

The hydrophilic polymer carrying multiple pendant sulphonyl groups, optionally with multiple pendant carboxylic groups, on each polymer molecule should be present at least at the lesion-contacting surface of the hydrogel composition. If desired, the hydrophilic polymer carrying multiple pendant sulphonyl groups, optionally with multiple pendant carboxylic groups, on each polymer molecule may also be present in the internal bulk of the composition, and/or a sulphonyl-free polymer or combination of polymers may be present in the internal bulk of the composition.

Generally, the degree of carboxylation of such a polymer is on average (number average) at least about one pendant carboxylic group per linear 100 carbon atoms of the carbon atom backbone of the polymer, for example up to about one pendant carboxylic group per linear six carbon atoms of the carbon atom backbone of the polymer.

The hydrogel used in the present invention suitably comprises a substantially water-insoluble, slightly crosslinked, partially neutralized, gel-forming polymer material having the pendant sulphonyl groups, and optionally pendant carboxylic groups, in acid or salt form at least at its lesion-contacting surface. Such polymer materials can be prepared from polymerizable, unsaturated, acid- and ester-containing monomers. Any polymer to be present at the lesion-contacting surface of the composition will contain pendant sulphonyl groups, for example —SO₃⁻ in acid or salt form, and optionally carboxylic groups in acid or salt form, as described herein. Thus, such monomers include the olefinically unsaturated acids, esters and anhydrides which contain at least one carbon to carbon olefinic double bond. More specifically, these monomers can be selected from olefinically unsaturated carboxylic acids, carboxylic esters, carboxylic acid anhydrides; olefinically unsaturated sulphonic acids; and mixtures thereof.

Olefinically unsaturated carboxylic acid, carboxylic acid ester and carboxylic acid anhydride monomers include the acrylic acids typified by acrylic acid itself, methacrylic acid, ethacrylic acid, α-chloroacrylic acid, α-cyano-acrylic acid, β-methyl-acrylic acid (crotonic acid), α-phenyl acrylic acid, β-acryloxy-propionic acid, sorbic acid, α-chloro-sorbic acid, angelic acid, cinnamic acid, p-chloro-cinnamic acid, β-styryl-acrylic acid (1-carboxy-4-phenyl-1,3-butadiene), itaconic acid, citraconic acid, mesaconic acid, glutaconic acid, aconitic acid, maleic acid, fumaric acid, tricarboxy-ethylene and maleic acid anhydride and salts (e.g. alkali metal salts such as sodium, potassium and lithium salts) thereof. For forming any polymer to be present at the lesion-contacting surface of the composition, the monomer or monomer mixture will include a monomer containing pendant sulphonyl groups, e.g. —SO₃⁻ in salt form counter balanced by sodium and or potassium and ammonium cations.

Olefinically unsaturated sulphonic acid monomers include aliphatic or aromatic vinyl sulphonic acids such as vinylsulphonic acid, allylsulphonic acid, vinyltoluenesulphonic acid and styrene sulphonic acid; vinyl sulphobetaines such as SPDA (1-propanaminium N,N-dimethyl-N-[2-[(1-oxo-2-propenyl)oxy]-3-sulfo hydroxide, inner salt (available from Raschig); acrylic and methacrylic sulphonic acid such as sulphoethyl acrylate, sulphoethyl methacrylate, suiphopropyl acrylate, sulphopropyl methacrylate, 2-hydroxy-3-acryloxy propyl sulphonic acid, 2-hydroxy-3-methacryloxy propyl sulphonic acid and 2-acrylamido-2-methyl-propanesulphonic acid and salts (e.g. ammonium or alkali metal salts, such as sodium, potassium and lithium salts, or alkaline earth metal salts, such as calcium or magnesium) thereof.

The monomers may suitably be used in admixture with each other or with other monomers. In one particularly useful embodiment of the invention, a monomer which has a first countercation associated with it may be used in admixture with one or more monomer which has/have one or more second/further countercation(s) associated with it/them. The monomers in their anionic form (i.e. disregarding the counter-cation) may be the same or different. In this way, the proportions of different cations (e.g. alkali metal ions such as sodium or potassium, or primary, secondary, tertiary or quaternary ammonium ions) can be finely controlled in the resultant polymer (homopolymer or copolymer), as previously discussed. The particular weight ratios of one monomer to the or each other monomer, and/or the respective countercations, can be selected within wide limits by those skilled in the art, depending on the desired properties of the resultant hydrogel polymer, and examples of suitable molar ratios have been given above in the Brief Description of the Invention.

Further examples of suitable monomers for use in the present invention include: a polyalkylene glycol acrylate or a substituted derivative thereof; a polyalkylene glycol methacrylate or a substituted derivative thereof; acrylic acid and salts thereof (e.g. alkali metal salts such as sodium, potassium and lithium salts); 2-acrylamido-2-methyl-propanesulphonic acid and salts thereof (e.g. ammonium or alkali metal salts, such as sodium, potassium and lithium salts, or alkaline earth metal salts, such as calcium or magnesium); acrylic acid (3-sulphopropyl) ester or a substituted derivative thereof or a salt thereof (e.g. an alkali metal salt such as sodium, potassium or lithium salt); diacetone acrylamide (N-1,1-dimethyl-3-oxobutyl-acrylamide); a vinyl lactam (e.g. N-vinyl pyrrolidone or a substituted derivative thereof); an optionally substituted N-alkylated acrylamide such as hydroxyethyl acrylamide; and an optionally substituted N,N-dialkylated acrylamide; and/or N-acryloyl morpholine or a substituted derivative thereof. For forming any polymer to be present at the lesion-contacting surface of the composition, the monomer or monomer mixture will include a monomer containing pendant sulphonyl groups, e.g. —SO₃⁻ in acid or salt form, and optionally carboxylic groups in acid or salt form.

The above monomers and monomer types may optionally include substituent groups. Optional substituents of the monomers used to prepare the hydrogels used in the present invention may preferably to selected from substituents which are known in the art or are reasonably expected to provide polymerisable monomers which form hydrogel polymers having the properties necessary for the present invention. Suitable substituents include, for example, lower alkyl, hydroxy, halo and amino groups.

In one particular form of the present invention, the hydrogel material may be free of uncrosslinked polymerised styrene sulphonates. In another particular form of the present invention, the hydrogel material may be free of any styrene sulphonate component, whether polymerised or unpolymerised and whether crosslinked or uncrosslinked.

The hydrogel used in the present invention preferably comprises a plasticised three-dimensional matrix of cross-linked polymer molecules, and preferably has sufficient structural integrity to be self-supporting even at very high levels of internal water content, with sufficient flexibility to conform to the surface contours of mammalian, preferably human, skin or other surface with which it is in contact.

The hydrogel generally comprises, in addition to the cross-linked polymeric network, an aqueous or non-aqueous plasticising medium including an organic plasticiser. This plasticising medium is preferably present in the same precursor solution as the monomer(s). The plasticising medium may comprise additional ingredients in solution or dispersion, as described in more detail below.

The hydrogel composition may suitably be present as a thin sheet, preferably supported by a sheet support member to provide mechanical strength. The sheet support member for the hydrogel may, for example, be a thin scrim or net structure, for example formed of a synthetic and/or natural polymer such as polyethylene or polypropylene. The sheet support member for the hydrogel may overlie the hydrogel sheet on the major face of the sheet directed away from the lesion in use, or may be embedded within the hydrogel polymer. The sheet support member may, if desired, extend beyond the margins of the hydrogel composition, and may be provided with a skin adhesive portion to secure the dressing to the skin. The skin adhesive portion may be hydrogel in nature (for example a plasticised tacky hydrogel, which may be the same as or different from the hydrogel provided on the support member for the treatment according to the present invention), or may be another type of skin adhesive selected from the many skin adhesives known in the wound dressings art. The support member may be or may comprise a sheet member as defined in WO 2007/113452, the contents of which is incorporated herein by reference. In particular, the support member may comprise or be a “fibrous absorbent sheet member” as defined in WO 2007/113452 and/or may comprise one or more other sheet members defined as “other absorbent sheet members” in WO 2007/113452. The dressing of the present invention may comprise an optional “net member” as defined in WO 2007/113452.

The hydrogel sheet may be part of a multi-layer composite, including further layers such as further hydrogels and/or other polymers and/or other sheet support members. For example, a breathable (air and/or moisture permeable) polymeric film (e.g. of polyurethane) may overlie the hydrogel sheet or composite on the major face of the sheet or composite directed away from the lesion in use.

The hydrogel composition and other sheet components as desired may preferably be provided with a release layer (e.g. of non-stick paper or plastic, such as siliconised paper or plastic) to protect one or both major face of the sheet prior to use.

The hydrogel composition and other sheet components as desired can constitute a dressing for the chronic ulcerous skin lesion which can, after removal of any release layer as appropriate, be applied to the lesion directly so that the major face which presents at its surface the hydrogel carrying pendant sulphonyl groups is directed towards the lesion and contacts the lesion, preferably the wound bed and surrounding tissues.

If desired, conventional bandages, cloths or other protective fabrics or materials can subsequently be applied to encase the dressing and hold it in place on the lesion.

Particularly where the hydrogel is plasticised, there is very slight adhesion between the hydrogel dressing and the patient's skin or the lesion tissue. This has the beneficial effect that one nurse or other healthcare professional can apply the dressing and can then prepare any desired bandages, cloths or the like for subsequent application. The dressing of the present invention will remain in place because of the mild adhesion, even if the patient moves before the further bandages etc. are applied.

The precursor liquid can comprise a solution of the gel-forming polymer in a relatively volatile solvent, whereby the hydrogel is deposited as a residue on evaporation of the solvent, or—more preferably—the precursor liquid will comprise a solution of the monomer(s), cross-linking agent, plasticiser, and optionally water and other ingredients as desired, whereby the hydrogel is formed by a curing reaction performed on the precursor liquid after application to the substrate to which the hydrogel is to be applied.

### Preparation of the Hydrogel and Dressing

In the following discussion, the second form of precursor solution and application protocol (in situ polymerisation of the hydrogel) will be discussed. The solvent deposition method carried out on a pre-formed gel-forming polymer is well known and the details of that procedure do not need to be reproduced here.

The polymerisation reaction is preferably a free-radical polymerisation with cross-linking, which may for example be induced by light, heat, radiation (e.g. ionising radiation), or redox catalysts, as is well known.

For example, the free radical polymerisation may be initiated in known manner by light (photoinitiation), particularly ultraviolet light (UV photoinitiation); heat (thermal initiation); electron beam (e-beam initiation); ionising radiation, particularly gamma radiation (gamma initiation); non-ionising radiation, particularly microwave radiation (microwave initiation); or any combination thereof. The precursor solution may include appropriate substances (initiators), at appropriate levels, e.g. up to about 5% by weight, more particularly between about 0.002% and about 2% by weight, which serve to assist the polymerisation and its initiation, in generally known manner.

Preferred photoinitiators include any of the following either alone or in combination:

Type I-α-hydroxy-ketones and benzilidimethyl-ketals e.g. Irgacure 651 (2,2-dimethoxy-2-phenylacetophenone). These are believed on irradiation to form benzoyl radicals that initiate polymerisation. Photoinitiators of this type that are preferred are those that do not carry substituents in the para position of the aromatic ring.

Preferred photoinitiators are 1-hydroxycyclohexyl phenyl ketone, for example as marketed under the trade name Irgacure 184 by Ciba Speciality Chemicals; Irgacure 651 (2,2-dimethoxy-2-phenylacetophenone); Darocur 1173 (2-hydroxy-2-propyl phenyl ketone); and mixtures of Irgacure 184 and Darocur 1173.

Photo-polymerisation is particularly suitable, and may be achieved using light, optionally together with other initiators, such as heat and/or ionising radiation. Photoinitiation will usually be applied by subjecting the pre-gel reaction mixture containing an appropriate photoinitiation agent to ultraviolet (UV) light. The incident UV intensity, at a wavelength in the range from 240 to 420 nm, is typically greater than about 10 mW/cm². The processing will generally be carried out in a controlled manner involving a precise predetermined sequence of mixing and thermal treatment or history.

The UV irradiation time scale should ideally be less than 60 seconds, and preferably less than 10 seconds to form a gel with better than 95% conversion of the monomers. Those skilled in the art will appreciate that the extent of irradiation will be dependent on a number of factors, including the UV intensity, the type of UV source used, the photoinitiator quantum yield, the amount of monomer(s) present, the nature of the monomer(s) present and the presence of polymerisation inhibitor.

The precursor solution (pre-gel) containing the monomer(s) and preferably cross-linking agent, water, plasticiser, photoinitiator and optionally other components as described below, is initially laid down on a substrate. Where the hydrogel composition is to be prepared in sheet for, the substrate will be a sheet. It may suitably comprise a release layer and any desired sheet support member (including, but not limited to, a non-woven or net structure) that may be interposed between the release layer and the hydrogel composition, or embedded withing the hydrogel composition, in the finished dressing. In this way, the precursor solution can be polymerised is situ on the release layer, preferably with all or substantially all other components of the final dressing in place.

In one preferred embodiment, (on the one hand) the precursor solution in contact with the substrate to which it is to be applied and (on the other hand) the source of the polymerisation initiator (e.g. the radiation source) may move relative to one another for the polymerisation step. In this way, a relatively large amount of polymerisable material can be polymerised in one procedure, more than could be handled in a static system. This moving, or continuous, production system is preferred.

After completion of the polymerisation, the product is preferably sterilised in conventional manner. The sterile composite may be used immediately, e.g. to provide a skin-adhesive layer in an article, or a top release layer may be applied to the composite for storage and transportation of the composite.

If desired, certain ingredients of the hydrogel may be added after the polymerisation and optional cross-linking reaction. However, it is generally preferred that substantially all of the final ingredients of the hydrogel are present in the precursor solution, and that—apart from minor conventional conditioning or, in some cases, subsequent modifications caused by the sterilisation procedure—substantially no chemical modification of the hydrogel takes place after completion of the polymerisation reaction.

Monomers

The monomers are discussed in more detail above. Particularly preferred monomers include: the sodium salt of 2-acrylamido-2-methylpropane sulphonic acid, commonly known as NaAMPS, which is available commercially at present from Lubrizol as either a 50% aqueous solution (reference code LZ2405) or a 58% aqueous solution (reference code LZ2405A); the potassium salt of 2-acrylamido-2-methylpropane sulphonic acid (Potassium AMPS), which is available commercially at present from Lubrizol; the ammonium salt of 2-acrylamido-2-methylpropane sulphonic acid (Ammonium AMPS), which is available commercially at present from Lubrizol; acrylic acid (3-sulphopropyl) ester potassium salt, commonly known as SPA or SPAK (SPA or SPAK is available commercially in the form of a pure solid from Raschig); acrylic acid (3-sulphopropyl) ester sodium salt, commonly known as SPANa (SPANa is available commercially in the form of a pure solid from Raschig); and SPDA. Acrylic acid (BASF) may be used as supplied or in partial or complete salt form where the salt counterion is an alkali metal (e.g. sodium or potassium), alkaline earth metal (e.g. calcium) or ammonium. Mixtures of any two or more of the above monomers may be used. When a mixture of the monomers is used, it may, for example, be a mixture of NaAMPS and SPAK, a mixture of NaAMPS and SPANa, a mixture of NaAMPS and Potassium AMPS, a mixture of NaAMPS and Ammonium AMPS, or a mixture of NaAMPS and acrylic acid. The relative amounts of the monomers in a mixture will be dictated by the desired ratio of counterions (e.g. potassium, sodium and ammonium) in the hydrogel, as well as the required properties of the copolymer, and may be selected easily by those skilled in the art, if necessary with routine testing of the copolymers prepared.

Cross-Linking Agents

Conventional cross-linking agents are suitably used to provide the necessary mechanical stability and to control the adhesive properties of the hydrogel. The amount of cross-linking agent required will be readily apparent to those skilled in the art such as from about 0.01% to about 0.5%, particularly from about 0.05% to about 0.4%, most particularly from about 0.08% to about 0.3%, by weight of the total polymerisation reaction mixture. Typical cross-linkers include tripropylene glycol diacrylate, ethylene glycol dimethacrylate, triacrylate, polyethylene glycol diacrylate (polyethylene glycol (PEG) molecular weight between about 100 and about 4000, for example PEG400 or PEG600), and methylene bis acrylamide.

Organic Plasticisers

The one or more organic plasticiser, when present, may suitably comprise any of the following either alone or in combination: at least one polyhydric alcohol (such as glycerol, polyethylene glycol, or sorbitol), at least one ester derived therefrom, at least one polymeric alcohol (such as polyethylene oxide) and/or at least one mono- or poly-alkylated derivative of a polyhydric or polymeric alcohol (such as alkylated polyethylene glycol). Glycerol is the preferred plasticiser. An alternative preferred plasticiser is the ester derived from boric acid and glycerol. When present, the organic plasticiser may comprise up to about 45% by weight of the hydrogel composition.

Surfactants

Any compatible surfactant may optionally be used as an additional ingredient of the hydrogel composition. Surfactants can lower the surface tension of the mixture before polymerisation and thus aid processing. The surfactant or surfactants may be non-ionic, anionic, zwitterionic or cationic, alone or in any mixture or combination. The surfactant may itself be reactive, i.e. capable of participating in the hydrogel-forming reaction. The total amount of surfactant, if present, is suitably up to about 10% by weight of the hydrogel composition, preferably from about 0.05% to about 4% by weight.

The surfactant may, for example, comprise at least one propylene oxide/ethylene oxide block copolymer, for example such as that supplied by BASF Plc under the trade name Pluronic P65 or L64.

Other Additives

The hydrogel in the composite of the present invention may include one or more additional ingredients, which may be added to the pre-polymerisation mixture or the polymerised product, at the choice of the skilled worker. Such additional ingredients are selected from additives known in the art, including, for example, water, organic plasticisers, surfactants, polymeric material (hydrophobic or hydrophilic in nature, including proteins, enzymes, naturally occurring polymers and gums), synthetic polymers with and without pendant carboxylic acids, electrolytes, osmolites, pH regulators, colorants, chloride sources, bioactive compounds and mixtures thereof. The polymers can be natural polymers (e.g. xanthan gum), synthetic polymers (e.g. polyoxypropylene-polyoxyethylene block copolymer or poly-(methyl vinyl ether alt maleic anhydride)), or any combination thereof. By “bioactive compounds” we mean any compound or mixture included within the hydrogel for some effect it has on living systems, whether the living system be bacteria or other microorganisms or higher animals such as the patient. Bioactive compounds that may be mentioned include, for example, pharmaceutically active compounds, antimicrobial agents, antiseptic agents, antibiotics and any combination thereof. Antimicrobial agents may, for example, include: sources of oxygen and/or iodine (e.g. hydrogen peroxide or a source thereof and/or an iodide salt such as potassium iodide) (see, for example Bioxzyme™ technology, for example in The Sunday Telegraph (UK) 26 Jan. 2003 or the discussion of the Oxyzyme™ system at www.wounds-uk.com/posterabstracts2003.pdf); honey (e.g. active Manuka honey); antimicrobial metals, metal ions and salts, such as, for example, silver-containing antimicrobial agents (e.g. colloidal silver, silver oxide, silver nitrate, silver thiosulphate, silver sulphadiazine, or any combination thereof), hyperchlorous acid; or any combination thereof.

In the Bioxzyme system, a dressing comprises two hydrogels. One contains glucose based antibacterial compounds and the other contains enzymes that convert the glucose into hydrogen peroxide. When these are exposed to air and contacted together at a wound site, the enzyme-containing gel being adjacent the skin and the glucose-containing gel overlying the enzyme-containing gel, a low level steady flow of hydrogen peroxide is produced, which inhibits anaerobic bacteria. This antibacterial effect can be enhanced by the inclusion of a very low level of iodide (less than about 0.04 wt %) in the hydrogel. The hydrogen peroxide and the iodide react to produce iodine, a potent antimicrobial agent.

Hydrogels incorporating antimicrobial agents may, for example, be active against such organisms as Staphylococcus aureus and Pseudomonas aeruginosa.

Agents for stimulating the healing of wounds and/or for restricting or preventing scarring may be incorporated into the hydrogel. Examples of such agents include growth factors such as TGF (transforming growth factor), PDGF (platelet derived growth factor), KGF (keratinocyte growth factor, e.g. KGF-1 or KGF-2), VEGF (vascular endothelial growth factor), IGF (insulin growth factor, optionally in association with one or more of IGF binding protein and vitronectin), e.g. from GroPep Ltd, Australia or Procyte, USA (see, e.g. WO-A-96/02270, the contents of which are incorporated herein by reference); cell nutrients (see, e.g., WO-A-93/04691, the contents of which are incorporated herein by reference); glucose (see, e.g., WO-A-93/10795, the contents of which are incorporated herein by reference); an anabolic hormone or hormone mixture such as insulin, triiodothyronine, thyroxine or any combination thereof (see, e.g., WO-A-93/04691, the contents of which are incorporated herein by reference); or any combination thereof.

Additional polymer(s), typically rheology modifying polymer(s), may be incorporated into the polymerisation reaction mixture at levels typically up to about 10% by weight of total polymerisation reaction mixture, e.g. from about 0.2% to about 10% by weight. Such polymer(s) may include polyacrylamide, poly-NaAMPS, polyethylene glycol (PEG), polyvinylpyrrolidone (PVP) or carboxymethyl cellulose.

Additional osmolite(s) may be included to modify the osmolarity of the hydrogel. Osmolites may be ionic (e.g. electrolytes, for example salts which are readily soluble in the aqueous phase of the hydrogel to increase the ionic strength of selected cations or anions and hence the osmolarity of the hydrogel). By selecting the ions present in an ionic osmolite, and particularly by selecting the cation so as to correspond or not with cationic counterions in the monomer(s) of the hydrogel, the ionic strength of certain anions (e.g. chloride) can be varied with fine control, without substantially changing the ionic strength of cations already present in very large amounts as counterions of the monomer(s).

Osmolites may be organic (non-ionic), for example organic molecules which dissolve in or intimately mix with the aqueous phase of the hydrogel to increase the osmolarity of the hydrogel deriving from non-ionic species in the aqueous phase. Such organic osmolites include, for example, water-soluble sugars (e.g. glucose and other monosaccharides), polyhydric alcohols (e.g. glycerol and other polyhydroxylated alkanols).

Additive ingredients may serve more than one purpose. For example, glycerol may serve as an organic plasticiser and an osmolite.

The hydrogel may comprise one or more complexing or chelating agents, which may include, but are not limited to, organic poly-carboxylic acids, and includes, but is not limited to, agents that can form complexes with or chelate to one or more metal ions. The complexing agent may be selected from di-, tri- and tetra-carboxylic acids. Preferably, the one or more complexing or chelating agents contain a moiety in which two carboxylic acid groups (CO₂H) or salts thereof are separated by three or four covalent bonds (e.g. three bonds in malic acid: (HO₂C)—CH₂—C H,OH—(CO₂H); four bonds in EDTA: (HO₂C)—CH₂—NR—C H₂—(CO₂H), in which R is the remaining part of the molecule). The complexing or chelating agents may comprise one or more molecules containing one or more primary, secondary or tertiary nitrogens within their structure. The complexing or chelating agents may include, but are not limited to, EDTA, citric acid, maleic acid, malic acid, and their salts (which include, but are not limited to, sodium and potassium salts). These agents have been found to be effective in controlling any ion exchange that may be associated with the hydrogel composition.

The hydrogel used in the present invention preferably consists essentially of a cross-linked hydrophilic polymer of a hydrophilic monomer and optionally one or more comonomer, together with water and/or one or more organic plasticiser, and optionally together with one or more additives selected from surfactants, polymers, pH regulators, electrolytes, osmolites, chloride sources, bioactive compounds and mixtures thereof, with less than about 40%, for example less than about 10%, by weight of other additives.

For further details of suitable hydrogel material for use in the present invention, and its preparation, please refer to the following publications: PCT Patent Applications Nos. WO-97/24149, WO-97/34947, WO-00/06214, WO-00/06215, WO-00/07638, WO-00/46319, WO-00/65143 and WO-01/96422, the disclosures of which are incorporated herein by reference.

The water activity, which is related to the osmolarity and the ionic strength of the precursor solution (as measured, for example, by a chilled mirror dewpoint meter, Aqualab T3) is preferably between 0.05 and 0.99, more preferably between, 0.2 and 0.99, and even more preferably between 0.3 and 0.98, for example between 0.6 and 0.89. The ionic strength of the precursor solution can therefore be used to optimise the hydrogel properties.

### Protease Modulation

The data and discussions included herein show that the present invention enables protease inhibition. When applied to wound treatment, and particularly to the treatment of chronic skin wounds of humans, this protease inhibition will assist the healing process, by breaking the cycle of protease activity which characterises poorly healing wounds (see discussion above in the “Background” section of this application).

The expression “protease modulation” and like expressions, used herein, refers to any enhancement or inhibition of protease enzyme activity. The modulator may, for example, be due to a suitable change in the concentration of the enzymes in the medium or a suitable change in the concentration of co-factors or inhibitors of the enzymes in the medium. Without being bound by theory, changes in concentration may, for example, be due to denaturing of the compounds or their absorption into the hydrogel composition. The change in concentration may be due to the adsorption of protease proteins and/or one or more constituent parts thereof onto the hydrogel composition and/or absorption of protease proteins and/or one or more Constituent parts thereof into the hydrogel composition.

The expression “protease enhancement” and the like expressions used herein, refers to any activation of protease enzymes. Protease enhancement would be considered substantial if the enzyme activity is at least doubled in comparison with the corresponding untreated protease-containing medium.

The expression “protease inhibition” and like expressions, used herein, refers to any deactivation of protease enzymes. Protease inhibition would be considered substantial if the enzyme activity is at least halved in comparison with a corresponding untreated protease-containing medium. Proteases that can be inhibited using the present invention include neutrophil-derived elastase, plasmin and matrix metalloproteins (MMPs), of which the MMPs may be particularly mentioned.

### BRIEF DESCRIPTION OF THE DRAWINGS

In the accompanying drawings:

FIG. 1 shows the gelatin zymography apparatus used in Example 12;

FIG. 2 shows the electrophoresis bands obtained in the gelatin zymography experiment described in Example 12;

FIGS. 3 to 5 show the results of the experiments to determine the absorption of a prototype matrix metalloprotease (MMP) by the hydrogel of Example 1 in the zymography experiment described in Example 12, as discussed in more detail in Example 13.

### EXAMPLES AND DETAILED DESCRIPTION OF THE DRAWINGS

The following non-limiting examples are provided as further illustration of the present invention, but without limitation.

In the following Examples, and throughout this description, parts and percentages are by weight unless otherwise stated.

### Examples 1 to 11

### Hydrogel Compositions

Examples 1 to 10 illustrate suitable hydrogel compositions which may be used with suitable sheet support members as described herein to provide a dressing for use in the present invention. Example 11 relates to a comparison material not according to the invention.

In these examples, each of the pre-gel formulations was cured as 0.3 to 2.6 kg per square metre coat weight by a medium pressure mercury arc lamp (GEW, UK).

### Example 1

Pre-gel: 67 parts by weight of 58% aqueous solution of the sodium salt of acrylamidomethyl-propanesulphonic acid (NaAMPS, LZ2405 Lubrizol), 3 parts acrylic acid (3-sulphopropyl) ester potassium salt, commonly known as SPA or SPAK (SPA or SPAK is available commercially in the form of a pure solid from Raschig), 20 parts water, 10 parts glycerol and 0.21 parts of a 1 to 10 (by weight) mixture of Daracure 1173 photoinitiator (Ciba Speciality Chemicals) and IRR280 cross-linker (PEG400 diacrylate, UCB Chemicals).

### Example 2

Pre-gel: 67 parts by weight of 58% aqueous solution of the sodium salt of acrylamidomethyl-propanesulphonic acid (NaAMPS, LZ2405 Lubrizol), 1 parts acrylic acid (3-sulphopropyl) ester potassium salt, commonly known as SPA or SPAK (SPA or SPAK is available commercially in the form of a pure solid from Raschig), 20 parts water, 10 parts glycerol and 0.21 parts of a 1 to 10 (by weight) mixture of Daracure 1173 photoinitiator (Ciba Speciality Chemicals) and IRR280 cross-linker (PEG400 diacrylate, UCB Chemicals).

### Example 3

Pre-gel: 67 parts by weight of 58% aqueous solution of the sodium salt of acrylamidomethyl-propanesulphonic acid (NaAMPS, LZ2405 Lubrizol), 0.5 parts acrylic acid (3-sulphopropyl) ester potassium salt, commonly known as SPA or SPAK (SPA or SPAK is available commercially in the form of a pure solid from Raschig), 20 parts water, 10 parts glycerol and 0.21 parts of a 1 to 10 (by weight) mixture of Daracure 1173 photoinitiator (Ciba Speciality Chemicals) and IRR280 cross-linker (PEG400 diacrylate, UCB Chemicals).

### Example 3A

Pre-gel: 67 parts by weight of 58% aqueous solution of the sodium salt of acrylamidomethyl-propanesulphonic acid (NaAMPS, LZ2405 Lubrizol), 0.5 parts acrylic acid (3-sulphopropyl) ester sodium salt, commonly known as sodium SPA or SPANa (SPANa is available in the form of a pure solid from Raschig), 20 parts water, 10 parts glycerol and 0.21 parts of a 1 to 10 (by weight) mixture of Daracure 1173 photoinitiator (Ciba Speciality Chemicals) and IRR280 cross-linker (PEG400 diacrylate, UCB Chemicals).

### Example 4

Pre-gel: 67 parts by weight of 58% aqueous solution of the sodium salt of acrylamidomethyl-propanesulphonic acid (NaAMPS, LZ2405 Lubrizol), 0.15 parts acrylic acid (3-sulphopropyl) ester potassium salt, commonly known as SPA or SPAK (SPA or SPAK is available commercially in the form of a pure solid from Raschig), 20 parts water, 10 parts glycerol and 0.21 parts of a 1 to 10 (by weight) mixture of Daracure 1173 photoinitiator (Ciba Speciality Chemicals) and IRR280 cross-linker (PEG400 diacrylate, UCB Chemicals).

### Example 5

Pre-gel: 67 parts by weight of 58% aqueous solution of the sodium salt of acrylamidomethyl-propanesulphonic acid (NaAMPS, LZ2405 Lubrizol), 0.5 parts acrylic acid (3-sulphopropyl) ester potassium salt, commonly known as SPA or SPAK (SPA or SPAK is available commercially in the form of a pure solid from Raschig), 10 parts water, 20 parts glycerol and 0.21 parts of a 1 to 10 (by weight) mixture of Daracure 1173 photoinitiator (Ciba Speciality Chemicals) and IRR280 cross-linker (PEG400 diacrylate, UCB Chemicals).

### Example 6

Pre-gel: 70 parts by weight of 58% aqueous solution of the sodium salt of acrylamidomethyl-propanesulphonic acid (NaAMPS, LZ2405 Lubrizol), 0.5 parts acrylic acid (3-sulphopropyl) ester potassium salt, commonly known as SPA or SPAK (SPA or SPAK is available commercially in the form of a pure solid from Raschig), 30 parts glycerol and 0.21 parts of a 1 to 10 (by weight) mixture of Daracure 1173 photoinitiator (Ciba Speciality Chemicals) and IRR280 cross-linker (PEG400 diacrylate, UCB Chemicals).

### Example 7

Pre-gel: 67 parts by weight of 58% aqueous solution of the sodium salt of acrylamidomethylpropanesulphonic acid (NaAMPS, Lubrizol), 3 parts by weight ammonium salt of acrylamidomethyl-propanesulphonic acid (NH3AMPS, Lubrizol), 20 parts glycerol, 10 parts water and 0.21 parts of a 1 to 10 (by weight) mixture of Daracure 1173 photoinitiator (Ciba Speciality Chemicals) and IRR280 cross-linker (PEG400 diacrylate, UCB Chemicals)

### Example 8

Pre-gel: 70 parts by weight of 58% aqueous solution of the sodium salt of acrylamidomethylpropanesulphonic acid (Na AMPS, LZ2405 Lubrizol), 30 parts glycerol and 0.14 parts of a 1 to 10 (by weight) mixture of Daracure 1173 photoinitiator (Ciba Speciality Chemicals) and IRR280 cross-linker (PEG400 diacrylate, UCB Chemicals)

### Example 9

Pre-gel: 70 parts by weight of 58% aqueous solution of the sodium salt of acrylamidomethyl-propanesulphonic acid (NaAMPS, LZ2405 Lubrizol), 20 parts water, 10 parts glycerol and 0.14 parts of a 1 to 10 (by weight) mixture of Daracure 1173 photoinitiator (Ciba Speciality Chemicals) and IRR280 cross-linker (PEG400 diacrylate, UCB Chemicals).

### Example 10

Pre-gel: 52 parts by weight of 58% aqueous solution of the sodium salt of acrylamidomethyl-propanesulphonic acid (NaAMPS, LZ2405 Lubrizol), 48 parts water and 0.14 parts of a 1 to 10 (by weight) mixture of Daracure 1173 photoinitiator (Ciba Speciality Chemicals) and IRR280 cross-linker (PEG400 diacrylate, UCB Chemicals).

### Example 11

### Comparison

By way of comparison, the commercially available non-ionic crosslinked hydrogel dressing Vigilon (from Bard) was used as Example 11.

### Example 12

### Gelatin Zymography

Gelatin substrate gel electrophoresis, or zymography, also known as sodium docecyl sulphate-polyacrylamide gel electrophoresis (SDS-PAGE), was performed as previously described (Tarlton J F, Bailey A J, Crawford E et al. Prognostic value of markers of collagen remodeling in venous ulcers. Wound Repair Regen 1999; 7: 347-55.). In this procedure, proteins are separated by size. The proteins are coated in SDS which gives them a negative charge, and migrate under electrophoresis through a thin gel, which separates them according to size (larger proteins being the slower moving).

The method used was briefly as follows.

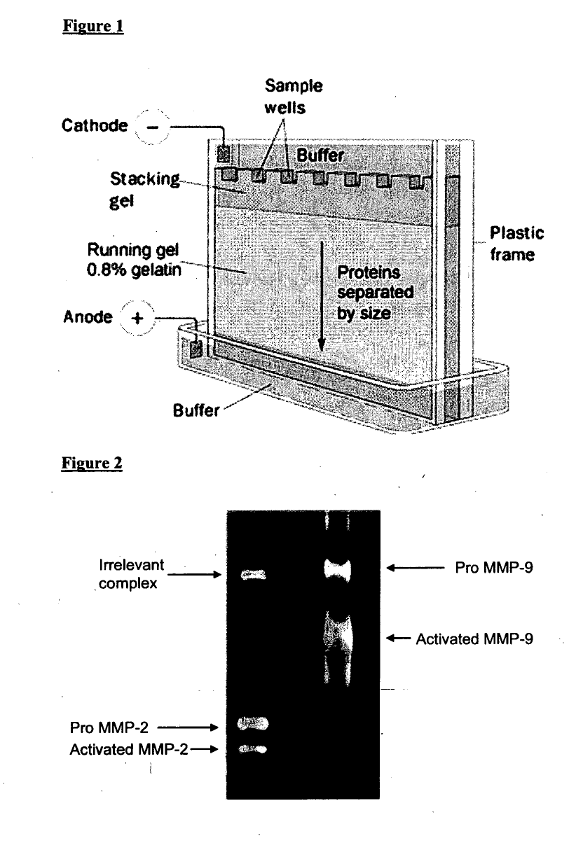

Immediately prior to polymerisation of a 10% sodium dodecyl sulphate (SDS)-polyacrylamide solution with 10% ammonium persulphate, warm gelatine solution was added to give a final gel concentration of 0.8% gelatine. Following polymerisation, a 6% stacking gel was added with a 15 sample well comb (see FIG. 1).

The matrix metalloproteases MMP-2 and MMP-9 (Calbiochem) were used as prototype MMPs in this study. 0.8 ng MMP-9 standard and 1.3 ng MMP-2 were diluted in an appropriate volume of non-reducing sample buffer, and loaded onto the gels which were run in a Biorad Mini Protean II gel system, at 20 mA per gel and 120v. Gels were washed for 10 minutes, 3 times in at least 5 times their own volume of 2.5% (v/v) Triton X-100 to displace SDS and allow enzyme renaturation. After a final wash in distilled water to remove traces of unwanted metal ions, the gels were incubated, with agitation at 37° C., for 16 hours in proteolysis buffer (50 mM Tris/HCl pH 7.8, 50 mM CaCl₂, 0.5 M NaCl), or proteolysis buffer treated as appropriate for dressing pre-treatment studies.

The gels were rinsed in water and stained with 0.1% Coomassie blue R250 in 16% acetic acid, 42% methanol and 42% water for at least one hour and destained in 7.5% acetic acid, 10% methanol and 82.5% water until the zones of proteolysis on the stacking gel had cleared. Densitometry was used to quantify the zones of proteolysis as previously described (Tarlton J F, Knight P J. Comparison of reflectance and transmission densitometry, using document and laser scanners, for quantitation of stained Western blots. Anal Biochem 1996; 237: 123-8).

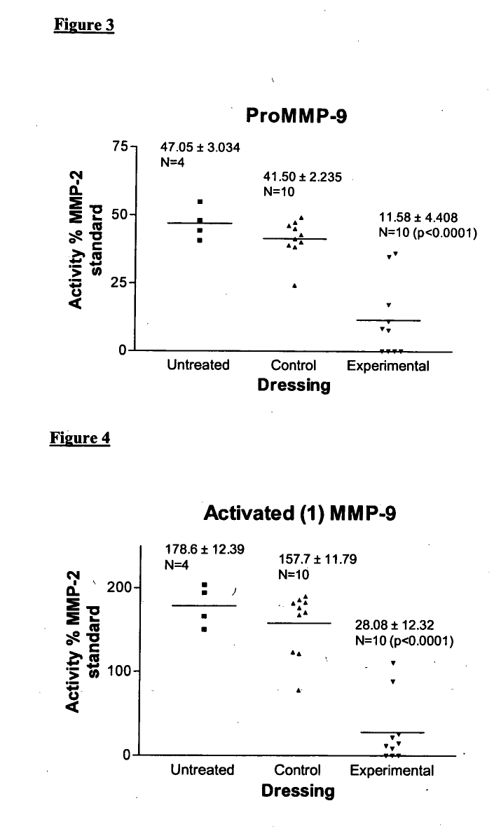

Following electrophoresis, proteolysis and staining, the undigested gelatine in the gel is seen as a dark (blue in reality) background, and the clear areas show sites of enzyme activity where the gelatine has been digested (see FIG. 2). For these experiments, purified enzymes were used so that two bands, representing pro- and activated enzyme, could be observed for each enzyme. Activated MMPs are generated in this system by interaction with SDS to produce a smaller molecule that travels further during electrophoresis.

Enzyme activity was quantified by measuring the clear area of gel at the expected location of each enzyme.

### Example 13

### MMP-9 Absorption by the Gels of Example 1 to 11

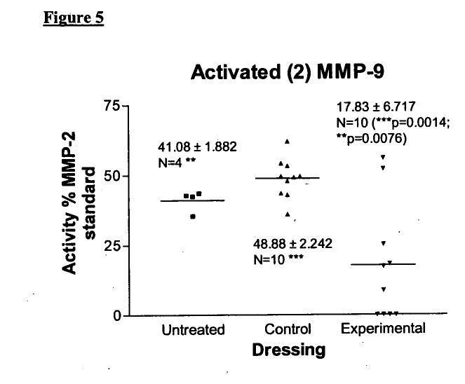

Ten mls of 100 ng/ml MMP-9 standard was prepared in 1/10 strength proteolysis buffer. At least 6 samples each of the tested gel and Vigilon (Comparison Example 11) were weighed out in aliquots of approximately 12 mg each. MMP-9 solution was added to each dressing aliquot at the rate of 33.3 ul per mg dressing and incubated at room temperature for 3 hours with mixing. After a short centrifugation, supernatant buffer was removed and 10 ul mixed with 10 ul sample buffer, and loaded onto zymogram SDS-PAGE gels at 10 and 3 ul per lane. After electrophoresis, the gels were incubated in proteolysis buffer, stained and scanned as described above (Example 12). The activity of the incubated samples was compared either to that of the untreated (non-incubated MMP-9 solutions), or to the activity of MMP-9 normalised to a standard of MMP-2 run at the same time on each gel. The results are described below with reference to FIGS. 3 to 5 of the accompanying drawings. The difference between the Figures is that the MMP-9 was alternatively used in the form of pro-MMP-9 (FIG. 3), or a first run using activated MMP-9 (FIG. 4), or a second run using activated MMP-9 (FIG. 5).

### Results

### MMP-9 Absorption by Example 1 and Vigilon

The ability of the gel made according to Example 1 (labelled “experimental dressing” in FIGS. 3 to 5) and Vigilon (labelled “control dressing” in FIGS. 3 to 5) to remove MMP-9 from solution was evaluated by incubation of each dressing with the protease for 3 hours.

Following zymography, it was clearly demonstrated that the activity of pro MMP-9 and activated MMP-9 in the Example 1 gel treated buffer solution was markedly decreased, in comparison to Vigilon which had little effect (see FIGS. 3 to 5).

The absorption experiment was repeated using the gels of Examples 2 to 10 in place of the gel of Example 1, and the results for all experiments are summarised in Table 1 below.

TABLE 1

| | Activity as % of untreated MMP-9 solution |
|---|---|
| Example | (average of at least 6 samples) |
| 1 | Less than 30% |
| 2 | Less than 30% |
| 3 | Less than 30% |
| 4 | Greater than 80% |
| 5 | Less than 30% |
| 6 | Less than 30% |
| 7 | Less than 50% |
| 8 | Greater than 80% |
| 9 | Greater than 80% |
| 10 | Greater than 50% |

Examples 1, 2, 3, 5, 6 and 7 relate to gels in which the molar ratio of sodium:potassium countercations and/or the ratio of “acrylamido-type” sulphonate groups:“acrylic acid sulphopropyl-type” sulphonate groups was less than 250:1. In all cases inhibition of protease enzymes (by which is meant a more than 50% reduction in activity compared with the untreated enzyme) was observed.

The other Examples relate to hydrogels in which the molar ratio of sodium:potassium countercations and/or the ratio of “acrylamido-type” sulphonate groups:“acrylic acid sulphopropyl-type” sulphonate groups was greater than 250:1. These produce a reduction in protease activity, but not so great as using the hydrogels of Examples 1, 2, 3, 5, 6 or 7.

The result of Example 7 shows that substituting ammonium ion for the sodium countercation maintains the protease inhibitory activity.

### INDUSTRIAL APPLICABILITY

The present invention provides an effective method of protease modulation, useful for example (but not exclusively) in the treatment of wounds, for example chronic skin lesions such as ulcerated skin lesions (e.g. chronic venous or arterial leg ulcers) to promote their healing.

In the context of the treatment of wounds, the method makes available protease modulation, and potentially simultaneous reduction of one or more undesirable characteristics of a wound, for example a chronic skin lesion, selected from pain associated with the wound, pain associated with changing of the dressing, exudation, malodour, irritation and hyperkeratosis, as has already been described in our PCT patent application No. PCT/GB2006/002632 (publication no. WO/2007/007115).

Undesirable effects of conventional dressings for wounds such as chronic skin lesions, for example maceration, incomplete absorption of exudate, excoriation, scarring of the final healed tissue, contact dermatitis, varicose eczema or skin stripping can also be reduced using the present invention in the context of wound treatment.

The hydrogel (dressing) used in the present invention is easy to apply and change, with resultant cost savings and efficiency enhancements.

Without wishing to be bound by theory, the hydrogel dressing is believed to mimic the natural extracellular matrix of a normal healing wound, and in particular certain sulphonated proteoglycans of the extracellular matrix such as heparin, using a moist wound healing environment where, in contrast to prior methods, the water levels are controlled to avoid the disadvantages of too much or too little moisture. The sulphonyl groups are believed to hold a relatively large hydration shell around them in the hydrogel, which may contribute to the very substantial wound healing and antimicrobial effects found with the hydrogels of the present invention.

The above broadly describes the present invention, without limitation. Variations and modifications as will be readily apparent to those of ordinary skill in this art are intended to be covered by this application and all subsequent patents.

1. A method of modulating proteases, comprising contacting a medium containing the proteases for an effective period of time with a hydrogel composition comprising a hydrophilic polymer carrying multiple pendant sulphonyl groups, optionally with multiple pendant carboxylic groups, on each polymer molecule.

2. A method according to claim 1, wherein the proteases are inhibited.

3. A method according to claim 1, wherein at least one protease is modulated.

4. A method according to claim 1, wherein matrix metalloprotease-9 (MMP-9) is inhibited.

5. A method according to claim 1, wherein the method is used in the treatment of a wound.

6. A method according to claim 5, wherein the wound is a skin wound.

7. A method according to claim 6, wherein the wound is a chronic ulcerous skin lesion.

8. A method according to claim 7, wherein the chronic ulcerous skin lesion is selected from venous leg ulcers, venous foot ulcers, arterial leg ulcers, arterial foot ulcers, decubitus ulcers (e.g. pressure sores, bedsores), post-surgical ulcerous lesions and chronic burn lesions.

9. A method according to claim 1 for modulating proteases in a wound, for example a chronic ulcerous skin lesion, in a human or non-human mammal, particularly a human, comprising contacting the wound for an effective period of time with a topical hydrogel composition comprising a hydrophilic polymer carrying multiple pendant sulphonyl groups, optionally with multiple pendant carboxylic groups, on each polymer molecule.

10. A method according to claim 9 for inhibiting proteases in a wound, for example a chronic ulcerous skin lesion, in a human or non-human mammal, particularly a human, comprising contacting the wound for an effective period of time with a topical hydrogel composition comprising a hydrophilic polymer carrying multiple pendant sulphonyl groups, optionally with multiple pendant carboxylic groups, on each polymer molecule.

11. A method according to claim 1, wherein in the hydrophilic polymer at least some of the pendant groups are present in salt form, so that charge-balancing countercations other than H⁺ are present in the hydrogel associated with the pendant groups.

12. A method according to claim 11, wherein two or more different countercations are present in the hydrogel.

13. A method according to claim 12, wherein the said countercations are selected from relatively weakly hydrated cations according to the Hofmeister series of cations, namely sodium or more weakly hydrated.

14. A method according to claim 13, wherein the two or more different countercations are selected from sodium, potassium, primary ammonium, secondary ammonium and tertiary ammonium cations.

15. A method according to claim 13, wherein the countercations are such that the first is the relatively more strongly hydrated according to the Hofmeister series of cations and the second is the relatively more weakly hydrated according to the Hofmeister series of cations.

16. A method according to claim 14, wherein the first cation is sodium and the second is selected from potassium, primary ammonium, secondary ammonium, tertiary ammonium and quaternary ammonium, or the first is potassium and the second is selected from primary ammonium, secondary ammonium, tertiary ammonium and quaternary ammonium.

17. A method according to claim 15, wherein the molar ratio of the first to the second counterions in the hydrophilic polymer is less than about 250:1, preferably less than about 200:1, for example less than about 100:1, for example less than about 80:1, for example less than about 50:1, and preferably more than about 2:1, for example, between about 2:1 and about 250:1, for example between about 5:1 and about 200:1, for example between about 5:1 and about 100:1, for example between about 7:1 and about 100:1, for example between about 10:1 and about 100:1, the first cation being the relatively more strongly hydrated according to the Hofmeister series of cations and the second being the relatively more weakly hydrated according to the Hofmeister series of cations.

18. A method according to claim 1, wherein the hydrophilic polymer is a homopolymer or copolymer comprising polymerised (co)monomer(s) carrying groups which provide the pendant groups of the polymer.

19. A method according to claim 18, wherein the monomer or monomers is/are selected from: the sodium salt of 2-acrylamido-2-methylpropane sulphonic acid (NaAMPS); the potassium salt of 2-acrylamido-2-methylpropane sulphonic acid (Potassium AMPS); the ammonium salt of 2-acrylamido-2-methylpropane sulphonic acid (Ammonium AMPS); acrylic acid (3-sulphopropyl) ester potassium salt (SPA or SPAK); acrylic acid (3-sulphopropyl) ester sodium salt (SPANa); SPDA; acrylic acid in partial or complete salt form where the salt counterion is an alkali metal (e.g. sodium or potassium), alkaline earth metal (e.g. calcium) or primary, secondary, tertiary or quaternary ammonium; and any combination or mixture of any two or more of the above.

20. A method according to claim 1, wherein the polymer is cross-linked.

21. A method according to claim 12, wherein the polymer is prepared by polymerising a first monomer in salt form comprising the first countercation and a second monomer, which may be the same as or different from the first monomer, in salt form comprising the second countercation, different from the first countercation.

22. A hydrogel composition comprising a hydrophilic homopolymer or copolymer carrying multiple pendant sulphonyl groups, optionally with multiple pendant carboxylic groups, the polymer comprising polymerised (co)monomer(s) each carrying groups which provide the pendant groups of the polymer, at least some of the said pendant groups of the polymer being in salt form with a first countercation and a second countercation, different from the first, wherein the said countercations are selected from relatively weakly hydrated cations according to the Hofmeister series of cations and the molar ratio of the said first to the said second countercations in the hydrophilic copolymer is less than about 250:1, preferably less than about 200:1, for example less than about 100:1, for example less than about 80:1, for example less than about 50:1, and preferably more than about 2:1, for example, between about 2:1 and about 250:1, for example between about 5:1 and about 200:1, for example between about 5:1 and about 100:1, for example between about 7:1 and about 100:1, for example between about 10:1 and about 100:1, the first cation being the relatively more strongly hydrated according to the Hofmeister series of cations and the second being the relatively more weakly hydrated according to the Hofmeister series of cations.

23. A hydrogel composition according to claim 22, wherein the said first and second countercations are selected from sodium, potassium, primary ammonium, secondary ammonium, tertiary ammonium and quaternary ammonium cations.

24. A hydrogel composition according to claim 23, wherein the first cation is sodium and the second is selected from potassium, primary ammonium, secondary ammonium, tertiary ammonium and quaternary ammonium, or the first is potassium and the second is selected from primary ammonium, secondary ammonium, tertiary ammonium and quaternary ammonium.

25. A hydrogel composition according to claim 22, for use in the treatment of a wound, for example a chronic skin lesion, in a human or non-human mammal, particularly a human.

26. A hydrogel composition for use as a protease modulator, particularly in the topical treatment of a wound, for example a chronic skin lesion, in a human or non-human mammal, particularly a human, the hydrogel composition comprising a hydrophilic polymer carrying multiple pendant sulphonyl groups, optionally with multiple pendant carboxylic groups.

27. A hydrogel composition according to claim 26 for use as a protease inhibitor, particularly in the topical treatment of a wound, for example a chronic skin lesion, in a human or non-human mammal, particularly a human, the hydrogel composition comprising a hydrophilic polymer carrying multiple pendant sulphonyl groups, optionally with multiple pendant carboxylic groups.

28. (canceled)

29. Use of a hydrogel composition comprising a hydrophilic polymer carrying multiple pendant sulphonyl groups, optionally with multiple pendant carboxylic groups, in the preparation of a topical medicament for use as a protease modulator in vivo, particularly in the treatment of a wound, for example a chronic skin lesion, in a human or non-human mammal, particularly a human.

30. (canceled)
